(19) United States
(12) Patent Application Publication
(10) Pub. No.: US 2026/0224855 A1
(43) Pub. Date: Aug. 6, 2026

(54) GUIDE CATHETER

(71) Applicant: Stallion Catheter, Inc., Redondo Beach, CA (US)

(72) Inventors: Himanshu Singh, Belmont, CA (US); Rushi Vrajmohan Parikh, Hermosa Beach, CA (US); Yohan Chacko, Graceville (AU); Andrew Fisher, Brooklyn Park, MN (US); Bruce Youngberg, Brooklyn Park, MN (US)

(21) Appl. No.: 19/150,888

(22) PCT Filed: Jan. 25, 2024
(86) PCT No.: PCT/US2024/012949

§ 371 Date: Jul. 24, 2025

Related U.S. Application Data
(60) Provisional application No. 63/481,598, filed on Jan. 25, 2023.

(51) Int. Cl. A61M 25/06; A61M 25/00; A61M 25/01; A61M 25/09

(52) U.S. Cl. CPC A61M 25/0662; A61M 25/0045; A61M 25/005; A61M 25/09; A61M 2025/0175

(57) ABSTRACT

A catheter system may include a guide catheter comprising a proximal end, a distal end, an inner liner, a braid surrounding the inner liner, and a jacket surrounding the braid. The catheter system may include a guide extension catheter comprising a guide extension wire and a tubular body, the tubular body of the guide extension catheter comprising a proximal end, a distal end, an inner liner, a coil surrounding the inner liner, and a jacket surrounding the coil. The guide catheter can be configured to receive the tubular body of the guide extension catheter. An inner diameter of the guide catheter decreases in the proximal direction, and an outer diameter of the tubular body increases in a distal direction. A proximal-most inner diameter of the guide catheter is smaller than a distal-most outer diameter of the tubular body.

### CROSS-REFERENCE TO RELATED APPLICATIONS

This application claims the benefit of U.S. Provisional Application No. 63/481598, entitled “GUIDE CATHETER” and filed on Jan. 25, 2023, which is hereby incorporated by reference in its entirety.

### TECHNICAL FIELD

The present invention relates generally to a guide extension catheter and/or a guide catheter including variable outer and/or inner diameters. The guide extension catheters and/or guide catheters can be used in cardiovascular procedures.

### BACKGROUND OF THE INVENTION

Embodiments described herein relate to catheter systems. The catheter system can include a guide catheter and a telescoping guide extension catheter. With catheters, inner diameter space is a premium, deployment into the vasculature a sensitive endeavor for safety reasons, and the time to deploy can also have severe safety and economic consequences. In current applications, guide extension catheters are commonly separate devices (from guide catheters) and are inserted into the guide catheter mid-procedure upon election by the practitioner. The current use of guide extension catheters can be cumbersome for physicians, waste time, and not optimize for inner diameter space. Embodiments of the catheter system disclosed herein can significantly improve upon these limitations by addressing these and other issues.

### SUMMARY

In some aspects, the techniques described herein relate to a catheter system for cardiovascular procedures. The catheter system can include a guide catheter including a proximal end, a distal end, an inner liner, a braid surrounding the inner liner, and a jacket surrounding the braid; a telescoping guide extension catheter including a guide extension wire and a tubular body, the tubular body of the telescoping guide extension catheter including a proximal end, a distal end, an inner liner, a coil surrounding the inner liner, and a jacket surrounding the coil; wherein the guide catheter is configured to receive the tubular body of the telescoping guide extension catheter; wherein an inner diameter of the guide catheter decreases in the proximal direction; wherein an outer diameter of the tubular body increases in a distal direction; wherein a proximal-most inner diameter of the guide catheter is smaller than a distal-most outer diameter of the tubular body.

The catheter system of any preceding paragraphs and/or any of the catheter systems disclosed herein can include one or more of the following features. In some aspects, the distal end of the tubular body can be configured to extend beyond the distal end of the guide catheter. The tubular body can include a plurality of segments, each of the plurality of segments including a durometer rating, where the durometer rating of each of the plurality of segments decreases in a distal direction. In some cases, an interior portion of the guide extension wire can be flush with an interior portion of the tubular body. The guide catheter can include a marker band positioned on a distal portion of the guide catheter. In some aspects, the telescoping guide extension catheter includes a marker band positioned on the tubular body. In some cases, the proximal-most inner diameter of the guide catheter can be between about 0.067 in and about 0.077 in, and the distal-most outer diameter of the tubular body can be between about 0.068 in and about 0.078 in. In some aspects, a difference between the proximal-most inner diameter of the guide catheter and the distal-most outer diameter of the tubular body is between about 0.005 in to about 0.05 in. An outer surface of the jacket of the tubular body can include a hydrophilic coating. In some cases, the inner liner of the guide catheter includes polytetrafluoroethylene (PTFE).

In some variants, the techniques described herein relate to a catheter system for cardiovascular procedures. The catheter system can include a guide catheter; a telescoping guide extension catheter including a guide extension wire and a tubular body; wherein the guide catheter is configured to receive the tubular body of the telescoping guide extension catheter; wherein a proximal-most inner diameter of the guide catheter is smaller than a distal-most outer diameter of the tubular body.

The catheter system of any preceding paragraphs and/or any of the catheter systems disclosed herein can include one or more of the following features. In some aspects, the guide catheter can include a proximal end, a distal end, an inner liner, a braid surrounding the inner liner, and a jacket surrounding the braid. In some cases, the tubular body of the telescoping guide extension catheter includes a proximal end, a distal end, an inner liner, a coil surrounding the inner liner, and a jacket surrounding the coil. The tubular body can include a plurality of segments, each of the plurality of segments including a durometer rating, where the durometer rating of each of the plurality of segments decreases in a distal direction. In some aspects, an inner diameter of the guide catheter can decrease in the proximal direction. The outer diameter of the tubular body can increase in a distal direction. In some cases, an interior portion of the guide extension wire can be flush with an interior portion of the tubular body. In some aspects, a difference between the proximal-most inner diameter of the guide catheter and the distal-most outer diameter of the tubular body can be between about 0.005 in to about 0.05 in. The catheter system can include a guide catheter and a guide extension catheter as shown and described herein.

### BRIEF DESCRIPTION OF THE DRAWINGS

FIG. 1A shows an example of a guide extension catheter.

FIG. 1B shows a cross-section view of the guide extension catheter shown in FIG. 1A taken along the line A-A shown in FIG. 1A.

FIG. 1C shows Detail B from FIG. 1B.

FIG. 2A shows cross-section view of an example of a guide catheter.

FIG. 2B shows Detail A from FIG. 2A.

FIG. 3A shows an example of a catheter system.

FIG. 3B shows a cross-section view of the catheter system shown in FIG. 3A taken along the line A-A shown in FIG. 3A.

FIG. 3C shows Detail B from FIG. 3B.

FIGS. 4A and 4B show an example of a guide catheter.

FIG. 5A shows an example of a guide extension catheter.

FIG. 5B shows a top view of the guide extension catheter shown in FIG. 5A.

FIG. 5C shows a side view of the guide extension catheter shown in FIGS. 5A and 5B.

### DETAILED DESCRIPTION

Catheter systems can include a guide catheter and a guide extension catheter. The guide extension catheter can extend within the guide catheter. Some of the major benefits of the catheter systems include maximizing inner diameter (for the passage of critical equipment needed for medical procedures), increased guide support to prevent backout of the catheter mid-procedure, and more consistent and smooth extension and retraction of the guide extension catheter. To maximize these benefits, the guide catheter and guide extension catheters can include variable wall thicknesses. The variable wall thicknesses can result in guide catheters and guide extension catheters of variable inner and/or outer diameters, specifically designed to operate with each other or to be nested together. Further details of guide catheters and guide extension catheters including variable inner and/out outer diameters are described in relation to FIGS. 30A-30F of International Application Number PCT/US2022/074029 (the PCT Application), which was filed on Jul. 21, 2022, and which is incorporated herein by reference in its entirety. The designs, figures, and ranges described herein can be used with 6 French catheters and systems. Although reference is made to dimensions relating to 6 French catheters and systems, the catheter components and systems described herein are not limited to 6 French or any specific dimensional range. A person of ordinary skill in the art can translate or scale these catheter designs and dimensions for other French sizes or sizes according to other standards.

The variable wall thickness of the guide catheter and/or the guide extension catheter can optimize inner diameter space and guide support. For example, the guide catheter and a tubular portion of the guide extension catheter can each include a distal end, a proximal end, a plurality of sections between the distal end and the proximal end. In such implementations, the guide catheter and the tubular body of the guide extension catheter can each have a varying wall thickness throughout the length of the device. The wall thickness can either lead to a variable inner diameter or a variable outer diameter of each catheter within the integrated catheter system. For example, the system can include a guide catheter with a constant outer diameter and a variable inner diameter. The system can include a guide extension catheter with a tubular body having a constant inner diameter and a variable outer diameter.

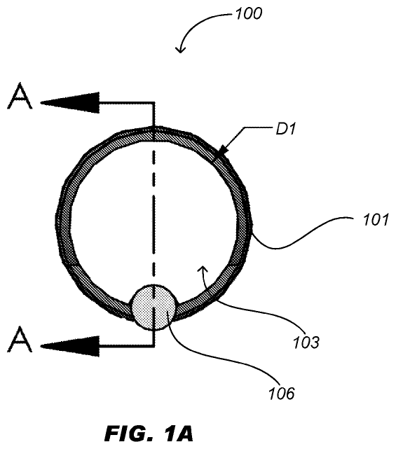

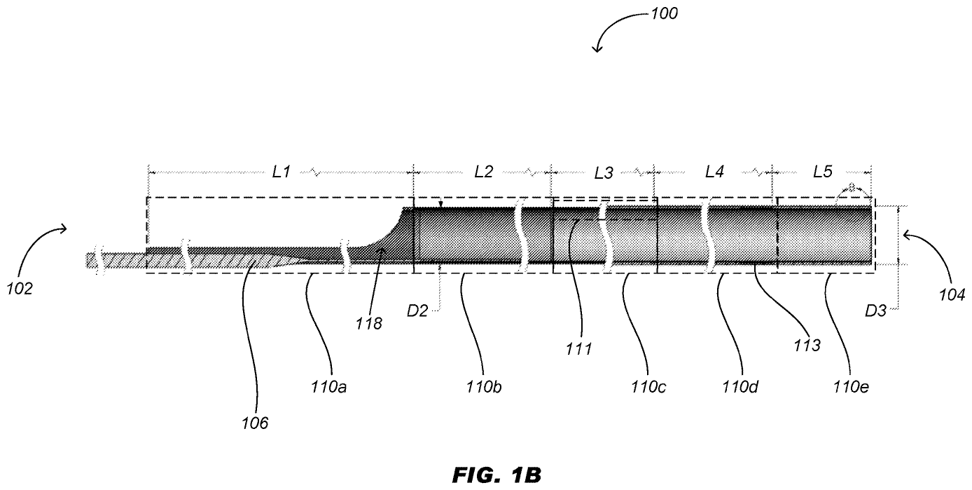

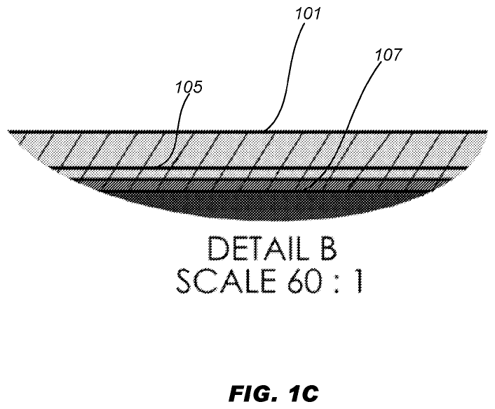

FIGS. 1A-1C show an example of a guide extension catheter. The guide extension catheter 100 can include a tubular body 101, a proximal portion 102 including a guide extension wire 106 and a distal portion 104 with a cylindrical braided and/or coiled section. The guide extension catheter 100 can include a transitional portion 118 that can allow a transitional feature between the proximal portion 102 including the guide extension wire 106 and the distal portion 104 with the cylindrical braided and/or coiled section. FIG. 1B illustrates a cross section of the guide extension catheter 100 taken along the line A-A shown in FIG. 1A. In some cases, the guide extension wire 106 can include a non-circular shaped portion and a circular shaped portion. In some cases, the guide extension wire 106 can include a non-circular shaped proximal portion than transitions to a circular shaped portion before being joined to the tubular body portion of the guide extension catheter. The design of the tubular body is critical for performance in many ways, including length, strength (radial or otherwise), pushability, lubricity, functional space, and various other well known catheter performance properties. The details described herein pertain to collective optimization of those factors.

As shown in FIG. 1B, the tubular body 101 of the guide extension catheter 100 can include a plurality of segments. For example, the tubular body 101 can include a first segment 110a, a second segment 110b, a third segment 110c, a fourth segment 110d, and a fifth segment 110e. Although reference is made to the tubular body 101 including five segments, the tubular body 101 can include less than or more than five segments (e.g., one, two, three, four, six, seven, eight segments, etc.).

The first segment 110a can include a length L1 that can be less than, equal to, and/or greater than 5 cm. In some cases, the length L1 can have a length of 2 cm to 8 cm (about 2 cm to about 8 cm). In some cases, the length L1 can have a length of 2 cm to 6 cm (about 2 cm to about 6 cm), 2 cm to 5 cm (about 2 cm to about 5 cm), 4 cm to 8 cm (about 4 cm to about 8 cm), 5 cm to 8 cm (about 5 cm to about 8 cm), or 6 cm to 8 cm (about 6 cm to about 8 cm).

The second segment 110b can include a length L2 that can be less than, equal to, and/or greater than 20 cm. In some cases, the length L2 can have a length of 10 cm to 30 cm (about 10 cm to about 30 cm). In some cases, the length L2 can have a length of 10 cm to 25 cm (about 10 cm to about 25 cm), 10 cm to 20 cm (about 10 cm to about 20 cm), 15 cm to 30 cm (about 15 cm to about 30 cm), 20 cm to 30 cm (about 20 cm to about 30 cm), or 25 cm to 30 cm (about 25 cm to about 30 cm).

The third segment 110c can include a length L3 that can be less than, equal to, and/or greater than 10 cm. In some cases, the length L3 can have a length of 5 cm to 15 cm (about 5 cm to about 15 cm). In some cases, the length L3 can have a length of 5 cm to 12 cm (about 5 cm to about 12 cm), 5 cm to 10 cm (about 5 cm to about 10 cm), 8 cm to 15 cm (about 8 cm to about 15 cm), 10 cm to 15 cm (about 10 cm to about 15 cm), or 12 cm to 15 cm (about 12 cm to about 15 cm).

The fourth segment 110d can include a length L4 that can be less than, equal to, and/or greater than 19.7 cm. In some cases, the length L4 can have a length of 10 cm to 30 cm (about 10 cm to about 30 cm). In some cases, the length L4 can have a length of 10 cm to 25 cm (about 10 cm to about 25 cm), 10 cm to 20 cm (about 10 cm to about 20 cm), 15 cm to 30 cm (about 15 cm to about 30 cm), 15 cm to 20 cm (about 15 cm to about 20 cm), 15 cm to 25 cm (about 15 cm to about 25 cm), 20 cm to 30 cm (about 20 cm to about 30 cm), or 25 cm to 30 cm (about 25 cm to about 30 cm).

The fifth segment 110e can include a length L5 that can be less than, equal to, and/or greater than 0.3 cm. In some cases, the length L5 can have a length of 0.1 cm to 0.5 cm (about 0.1 cm to about 0.5 cm). In some cases, the length L5 can have a length of 0.1 cm to 0.4 cm (about 0.1 cm to about 0.4 cm), 0.1 cm to 0.3 cm (about 0.1 cm to about 0.3 cm), 0.2 cm to 0.3 cm (about 0.2 cm to about 0.3 cm), 0.2 cm to 0.4 cm (about 0.2 cm to about 0.4 cm), 0.2 cm to 0.5 cm (about 0.2 cm to about 0.5 cm), 0.3 cm to 0.5 cm (about 0.3 cm to about 0.5 cm), or 0.4 cm to 0.5 cm (about 0.4 cm to about 0.5 cm). In some cases, a total length of the five segments 110a-110e of the guide extension catheter can be less than, equal to, and/or greater than 55 cm. In some cases, a total length of the five segments 110a-110e of the guide extension catheter 100 can be 55 cm (about 55 cm) or any length necessary to provide the needed guide catheter extension length and stability within the guide catheter system. In some cases, the guide extension catheter length can extend the entire length of a guide catheter.

The tubular body 101 can be extruded, molded, or made using any techniques for making catheter systems. For example, one or more sections 110a-110e of the tubular body 101 can be made of a thermoplastic elastomer. In some cases, distal segments of the tubular body 101 can be more flexible than more proximal segments of the tubular body 101. For example, and without limitation, the fifth segment 110e can be made of a thermoplastic elastomer extruded tube having a hardness of less than or at least 40D (on the Shore D Hardness Scale). In some cases, the first, second, third, and fourth segments 110a-110d can be made of a thermoplastic elastomer extruded tube having a hardness of less than or at least 72D (on the Shore D Hardness Scale).

In some cases, the guide extension catheter 100 includes a radiopaque band 113. The radiopaque band 113 can be disposed along a distal segment and/or a proximal segment of the tubular body 101. For example, the radiopaque band 113 can be disposed along the fourth segment 110d of the tubular body 101. The radiopaque band 113 can beneficially provide an indication about the location of the guide extension catheter 100 along a guide catheter or the vasculature of a patient.

In some cases, the tubular body 101 can be braided and. /or coiled for added stability. For example, and as shown in FIG. 1C, an interior 103 of the tubular body 101 can include a braided section 105. Additionally or alternatively, the tubular body 101 can include a coil. For example, the tubular body 101 can include both a braid and a coil, only a braid, and/or only a coil. The coil can include a stainless steel material. The braided section 105 can include a 0005″ X . 007″ braid with a braid density of less than or at least 70 PPI. The braided section 105 is not restricted to the braid density and braid type but can be any braid density and any braid type used for catheter systems. In some cases, the interior of the tubular body 101 can also include a liner 107. The braided section 105 can be disposed between the liner 107 and the interior of the tubular body 101. The braided section 105 and the liner 107 can extend along the entire length of the tubular body 101. In some cases, however, braided section 105 and the liner 107 extend along only a segment of the tubular body 101. For example, the braided section 105 and the liner 107 can extend only along the fifth segment 110e of the tubular body 101. In some cases, the liner 157 can be formed of PTFE or any material used for liners in catheter systems.

The tubular body 101 of the guide catheter 100 can include a variable wall thickness which can result in variable inner and/or outer diameters. In some cases, the wall thickness of the tubular body 101 can vary. For example, the tubular body 101 can include a constant inner diameter and a variable outer diameter. The tubular body 101 can include an inner diameter D1 of less than, equal to, and/or greater than 0.063 in. The inner diameter D1 can range from about 0.062 in to about 0.064 in (about 0.062 in to about 0.064 in), 0.062 in to 0.063 in (about 0.062 in to about 0.063 in), or 0.063 in to 0.064 in (about 0.063 in to about 0.064 in). In some cases, the outer diameter of the tubular body 101 can vary from a proximal portion 102 to the distal portion 104. In some cases, the tubular body 101 can include a transition region 111 where a wall thickness of the tubular body changes. The transition region 111 can extend along one or more of the segments 110a-110e. In some cases, the transition region 111 can extend along the third segment 110c. Depending on the length of the third segment 110c, the transition region can include a length ranging from about 5 cm to about 20 cm. A wall thickness of the tubular body can increase along the transition region 111 in a distal direction. This can result in an outer dimeter D2 of the proximal portion 102 of the tubular body 101 being smaller than an outer diameter D2 of the distal portion 104 of the tubular body 101. For example, the outer diameter D2 can be less than, equal to, and/or greater than 0.070 in and the outer diameter D3 can be less than, equal to, and/or greater than 0.073 in. In some cases, the outer diameter D2 can range from about 0.068 in to about 0.071 in (about 0.068 in to about 0.071 in), 0.068 in to 0.070 in (about 0.068 in to about 0.070 in), 0.068 in to 0.069 in (about 0.068 in to about 0.069 in), or 0.070 in to 0.071 in (about 0.070 in to about 0.071 in), and the outer diameter D3 can range from about 0.073 in to about 0.076 in (about 0.073 in to about 0.076 in), 0.073 in to 0.075 in (about 0.073 in to about 0.075 in), 0.073 in to 0.074 in (about 0.073 in to about 0.074 in), 0.074 in to 0.076 in (about 0.074 in to about 0.076 in), or 0.075 in to 0.076 in (about 0.075 in to about 0.076 in). Segments of the tubular body 101 adjacent to the transition region 111 can have constant wall thickness, which can result in a constant diameter. For example, the first and second segments 110a, 110b can have constant inner and outer diameters. Further, the fourth and fifth segments 110d, 110e can have constant inner and outer diameters. In some cases, the wall thickness on the proximal end of the transition region 111 can be the same or similar to the wall thickness of segment 110b and the wall thickness at the distal end of the transition region 111 can be the same or similar to the wall thickness of segment 110d.

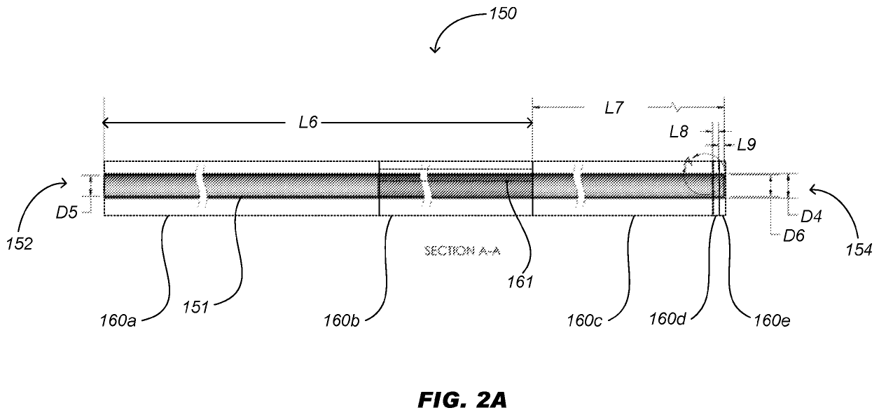

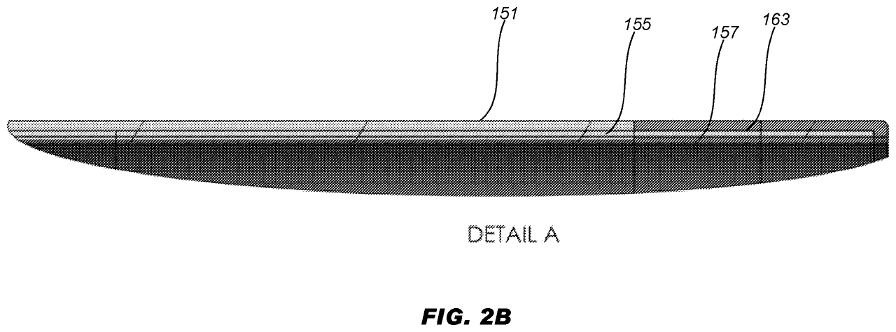

FIGS. 2A-2B show an example of a guide catheter. The guide catheter 150 can include a tubular body 151, a proximal portion 152 and a distal portion 154. The tubular body 151 of the guide catheter 150 can include a plurality of segments. For example, the tubular body 151 can include a first segment 160a, a second segment 160b, a third segment 160c, a fourth segment 160d, and a fifth segment 160e. Although reference is made to the tubular body 151 including five segments, the tubular body 151 can include less than or more than five segments (e.g., one, two, three, four, six, seven segments, etc.). A combined length L6 of the first and second segments 160a, 160b can be less than, equal to, and/or greater than 84 cm. In some cases, the length L6 can have a length of 60 cm to 100 cm (about 60 cm to about 100 cm). In some cases, the length L6 can have a length of 60 cm to 80 cm (about 60 cm to about 80 cm), 60 cm to 70 cm (about 2 cm to about 5 cm), 70 cm to 100 cm (about 70 cm to about 100 cm), 70 cm to 90 cm (about 70 cm to about 90 cm), 80 cm to 100 cm (about 80 cm to about 100 cm), 80 cm to 90 cm (about 80 cm to about 90 cm), or 90 cm to 100 cm (about 90 cm to about 100 cm).

The third segment 160c can include a length L7 that can be less than, equal to, and/or greater than 20 cm. In some cases, the length L7 can have a length of 10 cm to 30 cm (about 10 cm to about 30 cm). In some cases, the length L6 can have a length of 10 cm to 25 cm (about 10 cm to about 25 cm), 10 cm to 20 cm (about 10 cm to about 20 cm), 10 cm to 15 cm (about 10 cm to about 15 cm), 15 cm to 20 cm (about 15 cm to about 20 cm), 15 cm to 25 cm (about 15 cm to about 25 cm), 15 cm to 30 cm (about 15 cm to about 30 cm), 20 cm to 30 cm (about 20 cm to about 30 cm), or 25 cm to 30 cm (about 25 cm to about 30 cm).

The fourth segment 160d can include a length L8 that can be less than, equal to, and/or greater than 0.5 mm. In some cases, the length L8 can have a length of 0.2 cm to 0.8 cm (about 0.2 cm to about 0.8 cm). In some cases, the length L8 can have a length of 0.2 cm to 0.6 cm (about 0.2 cm to about 0.6 cm), 0.2 cm to 0.4 cm (about 0.2 cm to about 0.4 cm), 0.4 cm to 0.6 cm (about 0.4 cm to about 0.6 cm), 0.4 cm to 0.8 cm (about 0.4 cm to about 0.8 cm), or 0.6 cm to 0.8 cm (about 0.6 cm to about 0.8 cm).

The fifth segment 160e can include a length L9 that can be less than, equal to, and/or greater than 0.5 mm. In some cases, the length L9 can have a length of 0.2 cm to 0.8 cm (about 0.2 cm to about 0.8 cm). In some cases, the length L9 can have a length of 0.2 cm to 0.6 cm (about 0.2 cm to about 0.6 cm), 0.2 cm to 0.4 cm (about 0.2 cm to about 0.4 cm), 0.4 cm to 0.6 cm (about 0.4 cm to about 0.6 cm), 0.4 cm to 0.8 cm (about 0.4 cm to about 0.8 cm), or 0.6 cm to 0.8 cm (about 0.6 cm to about 0.8 cm). In some cases, a total length of the five segments 110a-110e of the tubular body 151 can be less than, equal to, and/or greater than 104 cm. In some cases, a total length of the five segments 110 a-110e of the guide catheter 150 can be 104 cm (about 104 cm) or any length necessary to provide the needed guide catheter length and stability within the guide catheter system or for the procedure. In some cases, the guide catheter length can extend the entire length of the guide extension catheter 100, and/or be greater than, or equal to the length of the guide extension catheter 100.

The tubular body 151 can be extruded, molded, or made using any techniques for making catheter systems. For example, one or more sections 160a-160e of the tubular body 151 can be made of a thermoplastic elastomer. In some cases, distal segments of the tubular body 151 can be more flexible than more proximal segments of the tubular body 151. For example, and without limitation, the fifth segment 160e can be made of a thermoplastic elastomer extruded tube having a hardness of less than or at least 40D (on the Shore D Hardness Scale). The fourth segment 160d can be made of a thermoplastic elastomer extruded tube having a hardness of less than or at least 55D. In some cases, the first, second, and third segments 160a-160c can be made of a thermoplastic elastomer extruded tube having a hardness of less than or at least 63D.

In some cases, the guide catheter 150 includes a radiopaque band 163, as shown in FIG. 2B. The radiopaque band 163 can be disposed along a distal segment of the tubular body 151. For example, the radiopaque band 163 can be disposed along at least one of the fourth and fifth segments 160d, 160e of the tubular body 151. The radiopaque band 163 can beneficially provide an indication about the location of the guide catheter 150 along the vasculature of a patient.

In some cases, the tubular body 151 can be braided for added stability. For example, and as shown in FIG. 2B, an interior portion of the tubular body 151 can include a braided section 155. In some cases, the braided section 155 can include a 0005″ X . 007″ braid with a braid density of less than or at least 70 PPI. The braided section 155 is not restricted to the braid density and braid type but can be any braid density and any braid type used for catheter systems. In some cases, the interior of the tubular body 151 can also include a liner 157. The braided section 155 can be disposed between the liner 157 and the interior portion of the tubular body 151. The braided section 155 and the liner 157 can extend along the entire length of the tubular body 151. In some cases, however, the braided section 155 and the liner 157 extend along only a segment of the tubular body 151. For example, the braided section 155 and the liner 157 can extend only along the third, fourth and fifth segments 160c, 160d, 160e of the tubular body 151. In some cases, the liner 157 can be formed of PTFE or any material used for liners in catheter systems.

The tubular body 151 of the guide catheter 150 can include a variable wall thickness which can result in variable inner and/or outer diameters. In some cases, the wall thickness of the tubular body 151 can vary. For example, the tubular body 151 can include a constant outer diameter and a variable inner diameter. The tubular body 151 can include an outer diameter D4 of less than, equal to, and/or greater than 0.082 in. The outer diameter D4 can range from about 0.081 in to about 0.083 in (about 0.081 in to about 0.083 in), 0.081 in to 0.082 in (about 0.081 in to about 0.082 in), or 0.082 in to 0.083 in (about 0.082 in to about 0.083 in). In some cases, the inner diameter of the tubular body 151 can vary from the proximal portion 152 to the distal portion 154. In some cases, the tubular body 151 can include a transition region 161 where a wall thickness of the tubular body changes. The transition region 161 can extend along one or more of the segments 160a-160e. In some cases, the transition region 161 can extends along the second segment 160b. The transition region 161 can include a length ranging from about 5 cm to about 20 cm. A wall thickness of the tubular body 151 can decrease along the transition region 161 in a distal direction. This can result in an inner dimeter D5 of the proximal portion 152 being smaller than an inner diameter D6 of the distal portion 154. For example, the inner diameter D5 can be less than, equal to, and/or greater than 0.072 in and the inner diameter D6 can be less than, equal to, and/or greater than 0.075 in. In some cases, the inner diameter D5 can range from about 0.070 in to about 0.072 in (about 0.070 in to about 0.072 in), 0.070 in to 0.071 in (about 0.070 in to about 0.071 in), or 0.071 in to 0.072 in (about 0.071 in to about 0.072 in), and the inner diameter D6 can range from about 0.074 in to about 0.078 in (about 0.074 in to about 0.078 in), 0.074 in to 0.076 in (about 0.074 in to about 0.076 in), . or 076 in to . 078 in (about . 076 in to about . 078 in). Segments of the tubular body 151 adjacent to the transition region 161 can have constant wall thickness, which can result in a constant diameter. In some cases, the wall thickness on the proximal end of the transition region 161 can be the same or similar to the wall thickness of segment 160a and the wall thickness at the distal end of the transition region 161 can be the same or similar to the wall thickness of segment 106c. For example, at least a portion of each segment 110a-110e can have constant inner and outer diameters.

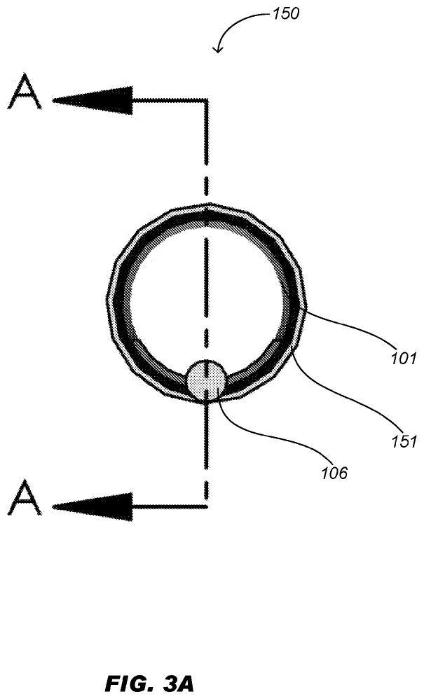

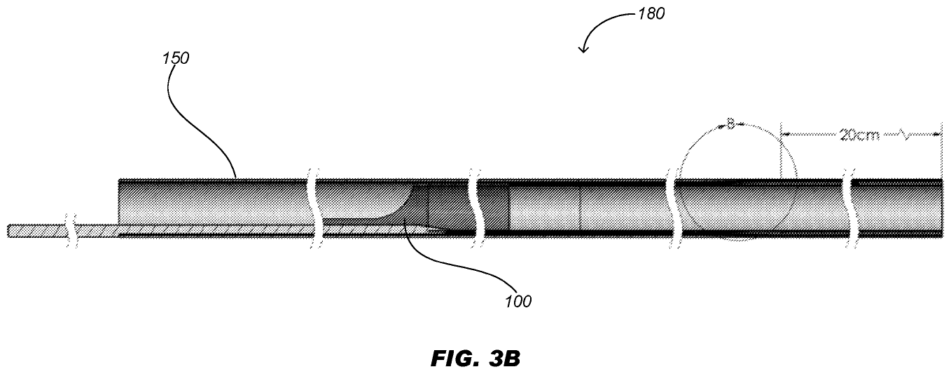

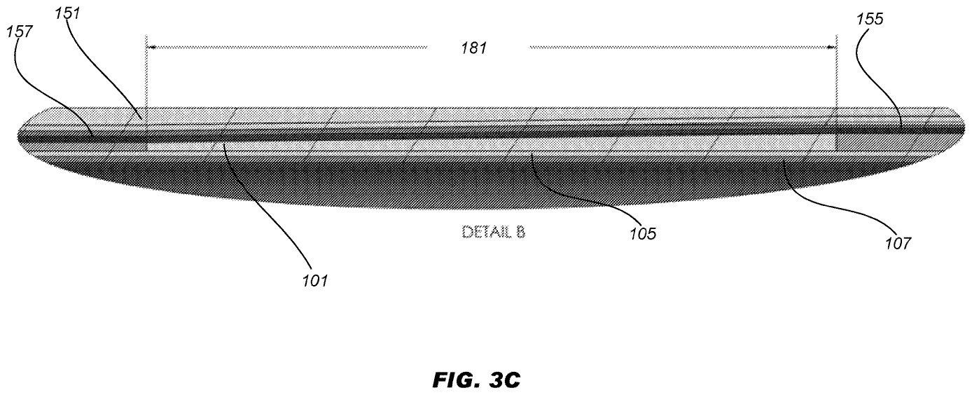

The guide extension catheter 100 and the guide catheter 150 can be combined to form a catheter system 180. For example, and as shown in FIG. 3B, the guide extension catheter 100 can extend through the guide catheter 150. To allow the guide extension catheter 100 to move within the guide catheter 150, the inner diameter of the guide catheter 150 can be larger than or as close as possible to the outer diameter of the guide extension catheter 100 along the length of the device. This arrangement can allow for a close-fitting concentric arrangement as shown in FIG. 3B while still allowing for the guide extension catheter 100 to be advanced and/retracted within the guide catheter 150 without resistance. In some cases, the guide extension catheter 100 can be extended beyond the distal portion 154 of the guide catheter 150.

The variable outer and/inner diameters of the guide extension catheter 100 and the guide catheter 150 can beneficially provide additional support as the guide extension catheter 100 extends further into the guide catheter 150. For example, an inner diameter at a proximal portion of the guide catheter 150 can be similar to an outer diameter at a distal portion of the guide extension catheter 100 therefore providing the guide support for the guide extension catheter. Further the variable thickness design optimizes for the maximum possible inner diameter of the guide extension catheter such that it does not have to be removed throughout the procedure-while not compromising the utility, feel, or use of the device. This improved guide support by allowing both catheters to be nested together throughout the procedure to prevent backout.

As shown in FIG. 3C, a wall thickness of the guide extension catheter 100 and the guide catheter 150 can taper along a transition region 181. In the transition region 181, an outer diameter of the guide extension catheter 100 increases in a proximal to distal direction as the thickness of the tubular body 101 increases in a proximal to distal direction, and an internal diameter of the guide catheter 150 increases in a proximal to distal direction as the thickness of the tubular body 151 decreases in a proximal to distal direction. The transition region 181 can have a length of less than, equal to, and/or greater than 10 cm. In some cases, the transition region 181 can have a length of 5 cm to 20 cm (about 5 cm to about 20 cm). In some cases, the transition region 181 can have a length of 5 cm to 15 cm (about 5 cm to about 15 cm), 5 cm to 10 cm (about 5 cm to about 10 cm), 10 cm to 15 cm (about 10 cm to about 15 cm), 10 cm to 20 cm (about 10 cm to about 20 cm), or 15 cm to 20 cm (about 15 cm to about 20 cm).

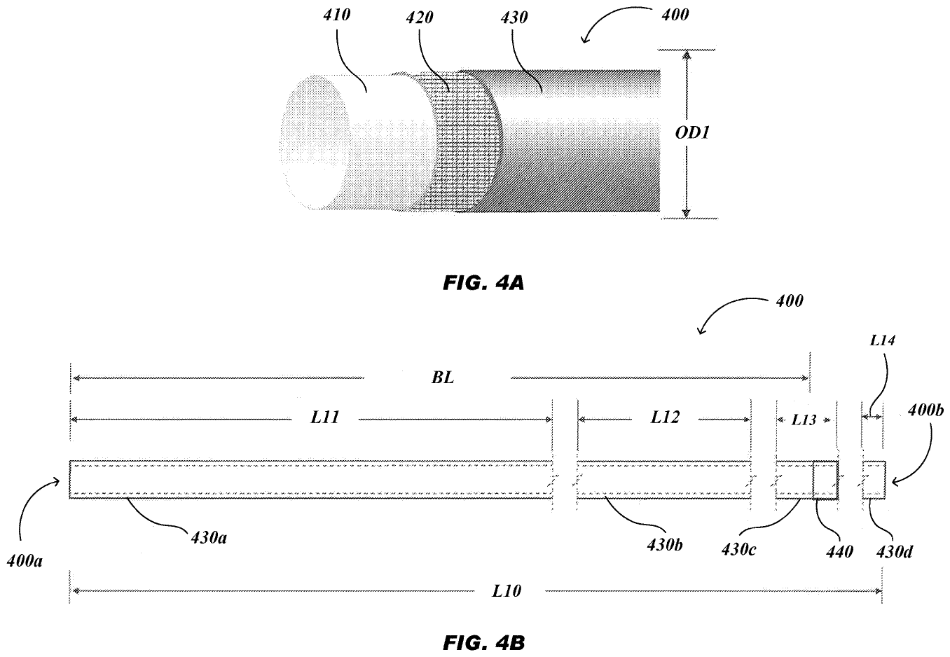

FIGS. 4A-4B show another example of a guide catheter 400. The guide catheter 400 can include a proximal end 400a, a distal end 400b, an inner liner 410, a braid 420 surrounding the inner liner, a jacket 430 surrounding the braid 420, and a marker band 440. In some cases, the guide catheter 400 can include a plurality of segments. For example, the guide catheter 400 can include a first segment 430a, a second segment 430b, a third segment 430c, and a fourth segment 430d. Although reference is made to the guide catheter 400 including four segments, the guide catheter 400 can include less than or more than four segments (e.g., one, two, three, five, six, seven segments, etc.). The braid 420 can extend the entire length of the guide catheter 400 but can also extend less than entire length of the guide catheter 400. For example, in some cases, the braid 420 can extend the entire length of the first segment 430a and the second segment 430b and a portion of the third segment 430c. As shown in FIG. 4B, a length BL of the braid 420 can extend along the guide catheter 400. In some cases, the braid 420 can extend from the first segment 430a to a proximal end of the marker band 440.

The guide catheter 400 be made of materials including Vestamid, Aesno, Pebax, polytetrafluoroethylene (PTFE), stainless steel, or a combination thereof. An exterior of the jacket 430 can include a hydrophilic coating. In some cases, the plurality of segments of the jacket 430 can include the same materials or different materials. For example, the first segment 430a can include a Vestamid material, the second segment 430b can include an Aesno material, and the third segment 430c and the fourth segment 430d can include a Pebax material. In some cases, the third segment 430c and the fourth segment 430d can include materials having different durometer ratings. For example, the third segment 430c can include a Pebax material having a durometer rating of less than, equal to, and/or greater than about 63D and the fourth segment 430 d can include a Pebax material having a durometer rating of less than, equal to, and/or greater than about 40D. Although reference is made to the third segment 430 c having a durometer rating of about 63D, the third segment 430 c can include a durometer rating of more than or less than 63D (e.g., 60D, 61D, 62D, 64D, 65D, 66D, etc.). Although reference is made to the fourth segment 430 d having a durometer rating of about 40D, the fourth segment 430 d can include a durometer rating of more than or less than 40D (e.g., 35D, 36D, 37D, 38D, 39D, 41D, 42D, 43D, 44D, 45D, etc.).

In some cases, the inner liner can be made of a PTFE material. The braid 420 can include a braid pic count of about 60. Although reference is made to the braid 420 having a braid pic count of about 60, the braid 420 can include a braid pic count of more than or less than 60 (e.g., 40, 45, 50, 55, 65, 70, 75, 80, etc.). The braid 420 can include a stainless steel wire with a thickness and width of about 0.007 in and 0.005 in respectively. In some cases, the marker band 440 can be made of platinum, iridium, or a combination thereof (e.g., Pt90/Ir10).

A combined length L10 of the segments 430a-430d can be less than, equal to, and/or greater than about 104 cm. In some cases, the length L10 can have a length of between about 60 cm to about 140 cm, between about 70 cm to about 130 cm, between about 80 cm to about 120 cm, between about 90 cm to about 110 cm, and/or between about 95 cm to about 105 cm. The guide catheter 400 can include an outer diameter OD1 that can be less than, equal to, and/or greater than about 0.082 in. In some cases, the outer diameter of the jacket 430 can be between about 0.040 in to about 0.12 in, between about 0.050 in to about 0.11 in, between about 0.060 in to about 0.10 in, between about 0.070 in to about 0.090 in, between about 0.075 in to about 0.085 in, and/or between about 0.0775 in to about 0.0825 in. The outer diameter OD1 can be constant throughout the entire length of the guide catheter 400.

In some cases, one of the inner diameter and the outer diameter OD1 of the guide catheter 400 can be constant and the other can be variable. For example, the outer diameter OD1 of the guide catheter 400 can be constant (e.g., the same) throughout an entire length of the guide catheter 400 and the inner diameter can be variable. In some cases, however, the inner diameter and/or the outer diameter OD1 of the guide catheter 400 can be different along the plurality of segments 430a-430d. For example, the inner diameter of the guide catheter 400 can increase in a proximal to distal direction so that the inner diameter along the first segment 430a is smaller than the inner diameter along the fourth segment 430d. In some cases, the inner diameter of the guide catheter 400 can decrease in a proximal to distal direction so that the inner diameter along the first segment 430a is larger than the inner diameter along the fourth segment 430d. The outer diameter OD1 of the guide catheter 400 can increase in a proximal to distal direction so that the outer diameter OD1 along the first segment 430a is smaller than the outer diameter along the fourth segment 430d. In some cases, the outer diameter OD1 of the guide catheter 400 can decrease in a proximal to distal direction so that the outer diameter OD1 along the first segment 430a is larger than the outer diameter OD1 along the fourth segment 430d. The thickness of the inner liner 410, the braid 420, and/or the jacket 430 can vary across the plurality of segments 430a-430d thus resulting in a guide catheter 400 with variable inner and/or outer diameters.

The first segment 430a of the guide catheter 400 can include a length L11 that can be less than, equal to, and/or greater than about 75 cm. In some cases, the length L11 can have a length of between about 50 cm to about 100 cm, between about 60 cm to about 90 cm, between about 65 cm to about 85 cm, and/or between about 70 cm to about 80 cm. The first segment 430a can include an inner diameter that can be less than, equal to, and/or greater than about 0.072 in. In some cases, the inner diameter of the first segment 430a can be between about 0.050 in to about 0.10 in, between about 0.06 in to about 0.09 in, between about 0.065 in to about 0.085 in, and/or between about 0.070 in to about 0.080 in.

The second segment 430b of the guide catheter 400 can include a length L12 that can be less than, equal to, and/or greater than about 27.5 cm. In some cases, the length L12 can have a length of between about 5 cm to about 50 cm, between about 10 cm to about 45 cm, between about 15 cm to about 40 cm, between about 20 cm to about 35 cm, and/or between about 25 cm to about 30 cm. The second segment 430b can include an inner diameter that can be less than, equal to, and/or greater than about 0.075 in. In some cases, the inner diameter of the second segment 430b can be between about 0.050 in to about 0.10 in, between about 0.060 in to about 0.090 in, between about 0.065 in to about 0.085 in, between about 0.070 in to about 0.080 in, and/or between about 0.0725 in to about 0.0775 in.

The third segment 430c of the guide catheter 400 can include a length L13 that can be less than, equal to, and/or greater than about 1 cm. In some cases, the length L13 can have a length of between about 0.2 cm to about 1.8 cm, between about 0.4 cm to about 1.6 cm, between about 0.6 cm to about 1.4 cm, between about 0.8 cm to about 1.2 cm, and/or between about 0.9 cm to about 1.1 cm. The third segment 430c can include an inner diameter that can be less than, equal to, and/or greater than about 0.075 in. In some cases, the inner diameter of the third segment 430c can be between about 0.050 in to about 0.10 in, between about 0.060 in to about 0.090 in, between about 0.065 in to about 0.085 in, between about 0.070 in to about 0.080 in, and/or between about 0.0725 in to about 0.0775 in.

The fourth segment 430d of the guide catheter 400 can include a length L14 that can be less than, equal to, and/or greater than about 0.5 cm. In some cases, the length L14 can have a length of between about 0.1 cm to about 0.9 cm, between about 0.2 cm to about 0.8 cm, between about 0.3 cm to about 0.7 cm, between about 0.4 cm to about 0.6 cm, and/or between about 0.45 cm to about 0.55 cm. The fourth segment 430d can include an inner diameter that can be less than, equal to, and/or greater than about 0.075 in. In some cases, the inner diameter of the fourth segment 430d can be between about 0.050 in to about 0.10 in, between about 0.060 in to about 0.090 in, between about 0.065 in to about 0.085 in, between about 0.070 in to about 0.080 in, and/or between about 0.0725 in to about 0.0775 in.

In some cases, the marker band 440 can be disposed along the one of the plurality of segments of the jacket 430. For example, the marker band 440 can be disposed along the fourth segment 430d of guide catheter 400. The marker band 440 can beneficially provide an indication about the location of the guide catheter 400 along the vasculature of a patient.

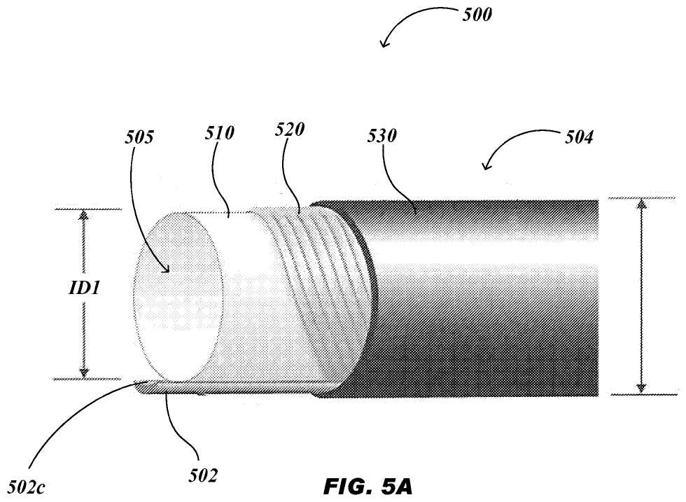

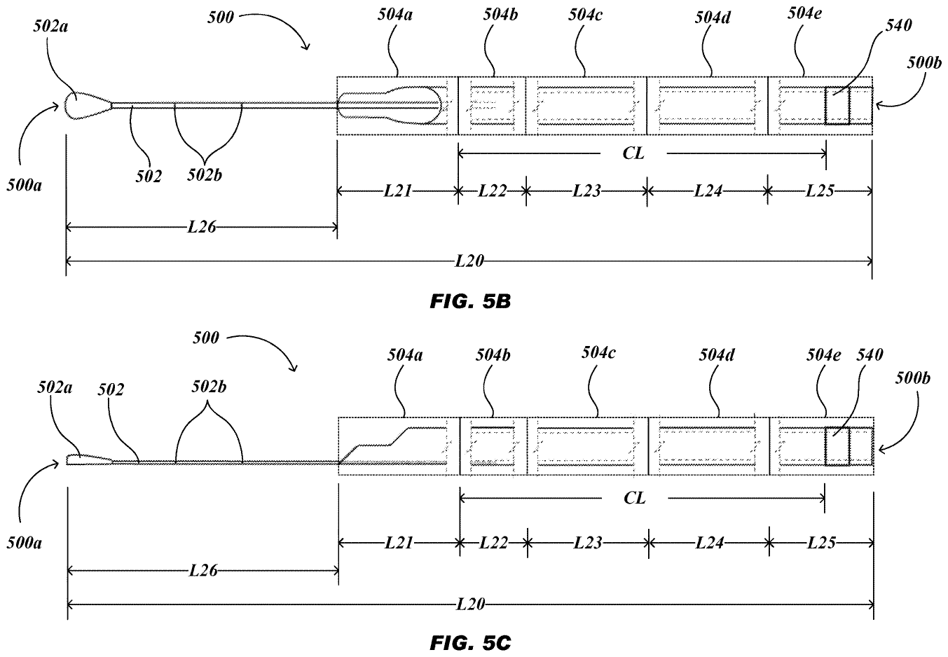

FIGS. 5A-5C show another example of a guide extension catheter 500 (also referred herein to as a telescoping guide extension catheter). The guide extension catheter 500 can include a proximal end 500a, a distal end 500b, a guide extension wire 502 (also referred herein to as a push rod), and a tubular body 504. A distal end of the guide extension wire 502 can be secured to the tubular body 504.

The guide extension wire 502 can include a tab 502a and one or more position markers 502b. The tubular body 504 can include an inner liner 510, a coil 520 surrounding the inner liner 510, a jacket 530 surrounding the coil 520, and a marker band 540. Additionally or alternatively, the tubular body 504 can include a braid. For example, the tubular body 504 can include both a braid and a coil, only a braid, and/or only a coil. In some cases, the tubular body 504 can include a plurality of segments. For example, tubular body 504 can include a first segment 504a, a second segment 504b, a third segment 504c, a fourth segment 504d, and a fifth segment 504e. Although reference is made to the tubular body 504 including five segments, the tubular body 504 can include less than or more than five segments (e.g., one, two, three, four, six, seven, eight, nine segments, etc.). The coil 520 can extend the entire length of the tubular body 504 but can also extend less than entire length of the tubular body 504. For example, in some cases, the coil 520 can extend along at least a portion of the second segment 504b, the third segment 504c, the fourth segment 504d, and/or the fifth segment 504e. As shown in FIGS. 5B-5C, a length CL of the coil 520 can extend along the tubular body 504. In some cases, the coil 520 can extend from a proximal end of the second segment 504b to a proximal end of the marker band 540. The CL can be less than, equal to, and/or greater than about 42.5 cm. In some cases, the length CL can have a length of between about 20 cm to about 60 cm, between about 30 cm to about 50 cm, between about 35 cm to about 45 cm, and/or between about 37 cm to about 43 cm.

The tubular body 504 can be made of materials including Vestamid, Acsno, Pebax, polytetrafluorocthylene (PTFE), stainless steel, or a combination thereof. An exterior of the jacket 530 can include a hydrophilic coating. In some cases, the plurality of segments of the tubular body 504 can include the same materials or different materials. For example, the plurality of segments of the tubular body can include different materials and/or different durometer ratings. For example, the plurality of segments 504a-504e can include a Pebax material. The first segment 504a and/or the second segment 504b can include a durometer rating of less than, equal to, and/or greater than about 72D. The third segment 504c can include a durometer rating of less than, equal to, and/or greater than about 55D. The fourth segment 504d can include a durometer rating of less than, equal to, and/or greater than about 40D. The fifth segment 504e can include a durometer rating of less than, equal to, and/or greater than about 35D.

Although reference is made to the plurality of segments of the tubular body 504 having a specific durometer, the plurality of segments can include a durometer rating of more than or less than that specified above. For example, the first segment 504a and/or the second segment 504b can include a durometer rating between about 62D and about 82D, between about 64D and about 80D, between about 66D and about 78D, between about 68D and 76D, between about 70D and about 74D, and/or between about 71D and 73D. The third segment 504c can include a durometer rating between about 45D and about 65D, between about 47D and about 63D, between about 49D and about 61D, between about 51D and about 59D, between about 53D and about 57D, and/or between about 54D and about 56D. The fourth segment 504d can include a durometer rating between about 30D and about 50D, between about 32D and about 48D, between about 34D and about 46D, between about 36D and 44D, between about 38D and about 42D, and/or between about 39D and about 41D. The fifth segment 504e can include a durometer rating between about 25D and about 45D, between about 27D and about 43D, between about 29D and about 41D, between about 31D and 39D, between about 33D and about 37D, and/or between about 34D and about 36D.

In some cases, the inner liner 510 can be made of a PTFE material. The coil 520 can include a stainless steel material. The coil 520 can include a width of less than, equal to, and/or greater than about 0.005 in, a pitch of less than, equal to, and/or greater than about 0.001 in, and/or a gap size of less than, equal to, and/or greater than about 0.012 in. Although reference is made to the coil 520 having specific width, pitch, and/or gap size, the coil 520 can include a width, pitch, and/or gap size of more than or less than those specified above. In some cases, the marker band 540 can be made of platinum, iridium, or a combination thereof (e.g., Pt90/Ir10). In some cases, the position markers 502b of the guide extension wire 502 can be laser marked into the guide extension wire 502.

In some cases, at least a portion of the guide extension wire 502 can be positioned between the coil 520 and the jacket 530, as shown in FIG. 5A. During a procedure, pushing and/or pulling the tab 502a and/or the guide extension wire 502 can beneficially cause the tubular body 504 to be advanced or retracted along the vasculature of a patient and/or another medical device (such as the guide catheter 400). In some cases, an interior portion 502c of the guide extension wire 502 can be flush or substantially flush with the inner liner 510. For example, the interior portion 502c can include a flat or substantially flat surface. The flat surface of the interior portion 502c can beneficially provide an unobstructed transition from the inner liner 510 to the guide extension wire 502, or vice versa. This can prevent the guide extension wire 502 from obstructing an interior portion 505 of the tubular body 504 which can be beneficial when advancing and/or retracting wires and/or other medical instruments through the tubular body 504. In some cases, the interior portion 502c can include a concave surface which can match the curvature of the interior portion 502c of the interior portion 505 of the tubular body 504 and/or the inner liner 510.

A combined length L20 of the guide extension wire 502 and the tubular body 504 can be less than, equal to, and/or greater than about 132 cm. In some cases, the length L20 can have a length of between about 90 cm to about 170 cm, between about 100 cm to about 160 cm, between about 110 cm to about 150 cm, between about 120 cm to about 140 cm, and/or between about 125 cm to about 135 cm. The tubular body 504 can include an inner diameter ID1 that can be less than, equal to, and/or greater than about 0.061 in. The inner diameter ID1 can between about 0.020 in to about 0.10 in, between about 0.030 in to about 0.090 in, between about 0.040 in to about 0.080 in, between about 0.050 in to about 0.070 in, and/or between about 0.055 in to about 0.065 in. The inner diameter ID1 can be constant throughout the entire length of the tubular body 504. In some cases, the outer diameter of the tubular body 504 can be between about 0.020 in to about 0.10 in, between about 0.030 in to about 0.090 in, between about 0.040 in to about 0.080 in, between about 0.050 in to about 0.070 in, between about 0.055 in to about 0.065 in, and/or between about 0.0575 in to about 0.0625 in.

In some cases, one of the inner diameter ID1 and the outer diameter of the tubular body 504 can be constant and the other can be variable. For example, the inner diameter ID1 of the tubular body 504 can be constant (e.g., the same) throughout an entire length of the tubular body 504 and the outer diameter can be variable. In some cases, however, the inner diameter ID1 and/or the outer diameter of the tubular body 504 can be different along the plurality of segments 504a-504e. For example, the inner diameter ID1 of the tubular body 504 can increase in a proximal to distal direction so that the inner diameter ID1 along the first segment 504a is smaller than the inner diameter ID1 along the fifth segment 504e. In some cases, the inner diameter ID1 of the tubular body 504 can decrease in a proximal to distal direction so that the inner diameter ID1 along the first segment 504a is larger than the inner diameter ID1 along the fifth segment 504e. The outer diameter of the tubular body 504 can increase in a proximal to distal direction so that the outer diameter along the first segment 504a is smaller than the outer diameter along the fifth segment 504e. In some cases, the outer diameter of the tubular body 504 can decrease in a proximal to distal direction so that the outer diameter along the first segment 504a is larger than the outer diameter along the fifth segment 504e. The thickness of the inner liner 510, the coil 520, and/or the jacket 530 can vary across the plurality of segments 504a-504e thus resulting in a tubular body 504 with variable inner and/or outer diameters.

The first segment 504a of the tubular body 504 can include a length L21 that can be less than, equal to, and/or greater than about 7.5 cm. In some cases, the length L21 can have a length of between about 5 cm to about 10 cm, between about 6 cm to about 9 cm, between about 6.5 cm to about 8.5 cm, and/or between about 7 cm to about 8 cm.

The second segment 504b of the tubular body 504 can include a length L22 that can be less than, equal to, and/or greater than about 2.5 cm. In some cases, the length L22 can have a length of between about 0.5 cm to about 4.5 cm, between about 1 cm to about 4 cm, between about 1.5 cm to about 3.5 cm, and/or between about 2 cm to about 3 cm. The second segment 504b can include an outer diameter that can be less than, equal to, and/or greater than about 0.070 in. In some cases, the outer diameter of the second segment 504b can be between about 0.030 in to about 0.11 in, between about 0.040 in to about 0.10 in, between about 0.050 in to about 0.090 in, between about 0.060 in to about 0.080 in, and/or between about 0.065 in to about 0.075 in.

The third segment 504c of the tubular body 504 can include a length L23 that can be less than, equal to, and/or greater than about 10 cm. In some cases, the length L23 can have a length of between about 5 cm to about 15 cm, between about 6 cm to about 14 cm, between about 7 cm to about 13 cm, between about 8 cm to about 12 cm, and/or between about 9 cm to about 11 cm. The third segment 504c can include an outer diameter that can be less than, equal to, and/or greater than about 0.070 in. In some cases, the outer diameter of the third segment 504c can be between about 0.030 in to about 0.11 in, between about 0.040 in to about 0.10 in, between about 0.050 in to about 0.090 in, between about 0.060 in to about 0.080 in, and/or between about 0.065 in to about 0.075 in.

The fourth segment 504d of the tubular body 504 can include a length L24 that can be less than, equal to, and/or greater than about 18 cm. In some cases, the length L24 can have a length of between about 10 cm to about 26 cm, between about 12 cm to about 24 cm, between about 14 cm to about 22 cm, between about 16 cm to about 20 cm, and/or between about 17 cm to about 19 cm. The fourth segment 504d can include an outer diameter that can be less than, equal to, and/or greater than about 0.070 in. In some cases, the outer diameter of the fourth segment 504d can be between about 0.030 in to about 0.11 in, between about 0.040 in to about 0.10 in, between about 0.050 in to about 0.090 in, between about 0.060 in to about 0.080 in, and/or between about 0.065 in to about 0.075 in.

The fifth segment 504e of the tubular body 504 can include a length L25 that can be less than, equal to, and/or greater than about 12 cm. In some cases, the length L25 can have a length of between about 5 cm to about 19 cm, between about 7 cm to about 17 cm, between about 9 cm to about 15 cm, between about 11 cm to about 13 cm, and/or between about 11.5 cm to about 12.5 cm. The fifth segment 504e can include an outer diameter that can be less than, equal to, and/or greater than about 0.073 in. In some cases, the outer diameter of the fifth segment 504e can be between about 0.050 in to about 0.10 in, between about 0.06 in to about 0.09 in, between about 0.065 in to about 0.085 in, between about 0.070 in to about 0.080 in, and/or between about 0.0725 in to about 0.0775 in.

A length L26 of the guide extension wire 502 from the proximal end 500a of the guide extension catheter 500 to a proximal end of the first segment 504a can be less than, equal to, and/or greater than about 82 cm. In some cases, the length L26 can have a length of between about 60 cm to about 105 cm, between about 70 cm to about 95 cm, between about 75 cm to about 90 cm, and/or between about 80 cm to about 85 cm.

In some cases, the marker band 540 can be disposed along the one of the plurality of segments of the tubular body 504. For example, the marker band 540 can be disposed along the fifth segment 504e of the tubular body 504. The marker band 540 can beneficially provide an indication about the location of the guide extension catheter 500 along the vasculature of a patient.

In some cases, the guide catheter 400 and the guide extension catheter 500 can be combined to form a catheter system. For example, the guide extension catheter 500 can extend through the guide catheter 400. To allow the guide extension catheter 500 to move within the guide catheter 400, an inner diameter of the guide catheter 400 can be larger than or as close as possible to the outer diameter of the guide extension catheter 500 along the length of the device. This arrangement can allow for a close-fitting concentric while still allowing the guide extension catheter 500 to be advanced and/retracted within the guide catheter 400 without resistance. In some cases, the guide extension catheter 500 can be extended beyond the distal end 400b of the guide catheter 400. In some cases, a proximal-most inner diameter of the guide catheter 400 can be smaller than a distal-most outer diameter of the tubular body 504 of the guide extension catheter 500. This can beneficially prevent the tubular body 504 from being completely retracted from the guide catheter 400 when the guide extension catheter 500 is positioned within the guide catheter 400.

The variable outer and/or inner diameters of the guide catheter 400 and the guide extension catheter 500 can provide the guide catheter 400 and/or the guide extension catheter 500 with a variable thickness. For example, as the outer and/or inner diameters of the guide catheter 400 and/or the guide extension catheter 500 increase or decrease, the thickness of the plurality of sections of the guide catheter 400 and/or the tubular body 504 may increase and/or decrease (e.g., the thickness may not be the same in all of the plurality of sections). This can beneficially provide additional support as the tubular body 504 of the guide extension catheter 500 extends further into the guide catheter 400. For example, an inner diameter at a proximal portion of the guide catheter 400 can be similar to an outer diameter at a distal portion of the guide extension catheter 500 so that the thickness of the guide catheter 400 and the tubular body 504 provide support for the guide extension catheter 500 when, for example, the guide tubular body 504 of the guide extension catheter 500 is fully nested within the guide catheter 400. In some cases, an outer diameter at a proximal portion of the guide extension catheter 500 can be similar to an inner diameter at a distal portion of the guide catheter 400 so that the thickness of the guide catheter 400 and the tubular body 504 provide support for the guide extension catheter 500 when, for example, the guide extension catheter 500 extends beyond the distal end 400b of the guide catheter 400. Further the variable thickness design optimizes for the maximum possible inner diameter of the guide extension catheter such that it does not have to be removed throughout the procedure—while not compromising the utility, feel, or use of the device. This improved guide support by allowing both catheters to be nested together throughout the procedure and/or prevent backout.

In some cases, the guide extension catheter 500 can be preloaded into the guide catheter 400 such that the tubular body 504 of the guide extension catheter 500 is positioned inside the extension catheter 400. The tubular body 504 can be translated along the guide between at least two positions by telescoping the tubular body 504 distally and/or proximally along the guide catheter 400, In a first position, the tubular body 504 can be retracted so that an entire length of the tubular body 504 is positioned within the guide catheter 400. In a second portion, the tubular body 504 can be extended so that at least a portion of the tubular body 504 extends beyond the distal end 400b of the guide catheter 400. In the second position, the variable thickness/outer diameter of the tubular body 504 and the thickness/inner diameter of the guide catheter 400 can reinforce and provide support along an entire length where the tubular body 504 and the guide catheter overlap.

For example, in some cases, the outer diameter of the guide catheter 400 can be the same or substantially the same along the entire length of the guide catheter 400 and the inner diameter of the guide tubular body 504 of the guide extension catheter 500 can be the same or substantially the same along the entire length of the guide tubular body 504 of the guide extension catheter 500. In such examples, as the inner diameter of the guide catheter 400 increases from the proximal end to the distal end of the guide catheter 400, the wall thickness of the guide catheter 400 can decrease from the proximal end to the distal end of the guide catheter 400. Additionally, as the outer diameter of the tubular body 504 of the guide extension catheter 500 increases from the proximal end to the distal end of the guide extension catheter 500, the wall thickness of the tubular body 504 increases from the proximal end to the distal end of the guide extension catheter 500. Accordingly, in the first position or nested configuration, the thinner wall thickness of the tubular body 504 can be aligned with the thicker wall thicknesses of the guide catheter 400 and the thinner wall thickness of the guide catheter 400 can be aligned with the thicker wall thicknesses of the tubular body 504. This can allow the device to have a similar stability throughout the entire length of the device regardless of the individual wall thickness at any portion of the guide catheter 400 and the guide extension catheter 500. Additionally, when the guide extension catheter 500 is in the second position and the guide extension catheter 500 is extended or telescoping from the guide catheter 400, the thinner wall thicknesses of the guide catheter 400 are in contact with the thinner wall thickness portions of the guide extension catheter 500 and the combined wall thickness can provide the necessary support for the device to allow movement and delivery of devices and instrumentation through the guide catheter 400 and the guide extension catheter 500.

Further details of guide catheters and guide extension catheters including variable inner and/out outer diameters can be found on International Application Number PCT/US2022/074029 (the PCT Application), which was filed on Jul. 21, 2022, and which is incorporated herein by reference in its entirety.

### Other Variations

Although some embodiments describe vascular procedures, the systems, devices, and/or methods disclosed herein can be applied to other types of therapies usable standalone or in addition to vascular procedures. The above figures and dimensions apply to coronary interventions, however, the innovations herein can be translated and applied for any other use, such as peripheral interventions.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. As one specific example, variable thickness can be applied to any variety of layers using any variety of materials, not just the one disclosed herein. A person of ordinary skill in the art can use various design alterations or manufacturing techniques within the novel variable thickness concepts disclosed. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A catheter system comprising:
a guide catheter comprising a proximal end, a distal end, an inner liner, a braid surrounding the inner liner, and a jacket surrounding the braid;
a telescoping guide extension catheter comprising a guide extension wire and a tubular body, the tubular body of the telescoping guide extension catheter comprising a proximal end, a distal end, an inner liner, a coil surrounding the inner liner, and a jacket surrounding the coil;
wherein the guide catheter is configured to receive the tubular body of the telescoping guide extension catheter;
wherein an inner diameter of the guide catheter decreases in the proximal direction;
wherein an outer diameter of the tubular body increases in a distal direction;
wherein a proximal-most inner diameter of the guide catheter is smaller than a distal-most outer diameter of the tubular body.

2. The catheter system of claim 1, wherein the distal end of the tubular body is configured to extend beyond the distal end of the guide catheter.

3. The catheter system of claim 1, wherein the tubular body comprises a plurality of segments, each of the plurality of segments comprising a durometer rating, where the durometer rating of each of the plurality of segments decreases in a distal direction.

4. The catheter system of claim 1, wherein an interior portion of the guide extension wire is flush with an interior portion of the tubular body.

5. The catheter system of claim 1, wherein the guide catheter comprises a marker band positioned on a distal portion of the guide catheter.

6. The catheter system of claim 1, wherein the telescoping guide extension catheter comprises a marker band positioned on the tubular body.

7. The catheter system of claim 1, wherein the proximal-most inner diameter of the guide catheter is between 0.067 in and 0.077 in.

8. The catheter system of claim 1, wherein the distal-most outer diameter of the tubular body is between 0.068 in and 0.078 in.

9. The catheter system of claim 1, wherein a difference between the proximal-most inner diameter of the guide catheter and the distal-most outer diameter of the tubular body is between about 0.005 in to about 0.05 in.

10. The catheter system of claim 1, wherein an outer surface of the jacket of the tubular body comprises a hydrophilic coating.

11. The catheter system of claim 1, wherein the inner liner of the guide catheter comprises polytetrafluoroethylene (PTFE).

12. A catheter system for cardiovascular procedures, the catheter system comprising:
a guide catheter;
a telescoping guide extension catheter comprising a guide extension wire and a tubular body;
wherein the guide catheter is configured to receive the tubular body of the telescoping guide extension catheter;
wherein a proximal-most inner diameter of the guide catheter is smaller than a distal-most outer diameter of the tubular body.

13. The catheter system of claim 12, wherein the guide catheter comprises a proximal end, a distal end, an inner liner, a braid surrounding the inner liner, and a jacket surrounding the braid.

14. The catheter system of claim 12, wherein the tubular body of the telescoping guide extension catheter comprises a proximal end, a distal end, an inner liner, a coil surrounding the inner liner, and a jacket surrounding the coil.

15. The catheter system of claim 14, wherein the tubular body comprises a plurality of segments, each of the plurality of segments comprising a durometer rating, where the durometer rating of each of the plurality of segments decreases in a distal direction.

16. The catheter system of claim 12, wherein an inner diameter of the guide catheter decreases in the proximal direction.

17. The catheter system of claim 12, wherein an outer diameter of the tubular body increases in a distal direction.

18. The catheter system of claim 12, wherein an interior portion of the guide extension wire is flush with an interior portion of the tubular body.

19. The catheter system of claim 12, wherein a difference between the proximal-most inner diameter of the guide catheter and the distal-most outer diameter of the tubular body is between about 0.005 in to about 0.05 in.

20. (canceled)
